(19) United States
(12) Patent Application Publication
(10) Pub. No.: US 2008/0304012 A1
(43) Pub. Date: Dec. 11, 2008

(54) RETINAL REFLECTION GENERATION AND DETECTION SYSTEM AND ASSOCIATED METHODS

(76) Inventors: Young K. Kwon, Oviedo, FL (US); Ceyhun Akcay, Mansfield, TX (US)

Correspondence Address: ALCON, IP LEGAL, TB4-8, 6201 SOUTH FREEWAY, FORT WORTH, TX, 76134, US

(21) Appl. No.: 11/758,944

(22) Filed: Jun. 6, 2007

(51) Int. Cl. A61B 3/11

(52) U.S. Cl. 351/221

(57) ABSTRACT

A system for generating a beam for retinal reflection detection includes a beam processor positioned to receive an illumination beam having a first spot size from a light source. The beam processor can alter the illumination beam to form a beam having a second spot size. Focusing optics focus this altered beam onto an eye, causing a spot to be formed on the retina. A detector receives reflected radiation from the retina passed through the pupil, and generates data indicative of a spatial extent of the eye pupil and an intensity map of the reflected radiation. A software package can determine from the data a pupil size and an intensity level in the intensity map. A controller in communication with the software signals the beam processor to vary the second spot size to optimize an accuracy of the determined pupil size and the intensity level.

### FIELD OF THE INVENTION

The invention relates generally to optical tracking systems, and more particularly to optical tracking systems that illuminate a retina for producing a reflection for tracking pupil position and size.

### BACKGROUND OF THE INVENTION

In an ophthalmic surgical procedure, unwanted eye movement can degrade the outcome of the surgery. Eye positioning is critical in such procedures as corneal ablation, since a treatment laser is typically centered on the patient's theoretical visual axis which, practically speaking, is approximately the center of the patient's pupil. However, this visual axis is difficult to determine due in part to residual and involuntary eye movement. Therefore, for best outcomes it is critical to stabilize the eye with respect to the surgical apparatus. It is particularly critical to stabilize the eye when using a small-spot refractive surgery system. Eye stabilization is typically performed with the use of an eye tracker.

Previous disclosure of eye tracking systems and methods has been made, for example, in U.S. Pat. Nos. 5,980,513; 6,315,773; and 6,451,008, which are co-owned with the present application, and which are hereby fully incorporated by reference hereinto. Video and LADAR tracking are also known in the art. Most known systems for tracking an eye require a specular reflection from the cornea as a reference, which cannot be used in LASIK-type surgeries, since the smooth surface of the cornea is replaced with a rougher surface when the stroma is exposed by flap cutting. Video trackers have been shown to work for this purpose, but these are not robust for eyes having a pupil size much smaller than the size of the rough flap surface, owing to a blurred video image of the pupil and iris in such cases. Further, these prior art systems tend to be relatively expensive, as they require high-speed cameras and high-speed processing capabilities. In addition, the trackers known to be used at the present time are not known to be successful with small, undilated pupils and with eyes implanted with intraocular lenses.

Current video-based trackers using pupil glow are designed to place a light spot at the retina that is either as broad as possible or as small as possible by focusing the light spot on the retina. Either of these extremes can cause significant tracking errors by forming pupil glow images with missing image rays at an incorrect size, or by an inability to form images with sufficient contrast between the pupil and the iris. Further missing “pupil edge” rays in the glow image are more often observed with highly myopic or hyperopic eyes.

The numerical aperture of the tracking system focusing optics determines the spot size at the retina. A larger spot size requires focusing optics designed to provide higher-numerical-aperture focusing on the cornea, which decreases the intensity of the light diffusely back-reflected and scattered at the vitreous/retina interface and retinal layers. If the intensity is too small, the pupil glow image can disappear below the camera's noise level, which disables a tracking operation. A smaller spot size (for example, <1 mm) can produce an incorrect pupil glow image size if the rays passing through the pupil edge are not able to form an image at the tracker camera, and serious tracking errors can also occur.

Therefore, it would be desirable to provide a system and method for tracking eyes, for example, during a surgical procedure, that do not rely on particular corneal properties, and that are also capable of effectively functioning on pupils in an undilated condition. It would further be desirable to provide an eye tracking system and method that can optimize spot size for minimizing tracking errors using pupil glow imaging.

### SUMMARY OF THE INVENTION

The embodiments of the present invention are useful for tracking eye movement by using a detector and the eye's retroreflecting properties, and can be used effectively on dilated and undilated eyes. The embodiments of the present invention can detect pupil “glow,” which is unfocused radiation projected onto and reflected by the retina, and detected on the pupil. Here the back-illuminated pupil boundary is seen via unfocused light reflected from the retina. Thus, there should be substantially no data impinging on the detector relating to external eye structure or features other than pupil size. Ideally, the radiation reflected should form a step function, with all radiation received at the detector from the pupil and the area surrounding the pupil contributing no data. In reality, of course, it is difficult to achieve a completely “on/off” data set, and the spot size and intensity have an effect on the sharpness of the pupil boundary. There are safety limits, however, on the intensity of the beam that can be projected onto and into the eye, and therefore the embodiments of the system and method of the present invention seek to optimize the intensity of the beam reflected from the retina by altering the impinging spot size.

One embodiment of the system for generating a beam for retinal reflection detection of this invention comprises a beam processor positioned to receive an illumination beam from a light source. The beam has a first spot size. The beam processor comprises means for altering the illumination beam to emit a beam having a second spot size. Focusing optics are positioned to receive the beam emitted from the beam processor and to focus the emitted beam onto an eye. A focal point of this emitted beam is adjacent a center of a pupil of an eye, thus causing a light spot to be formed on a retina of the eye.

A detector is adapted to receive reflected radiation from the retina passed through the pupil, and to generate data indicative of a spatial extent of the eye pupil and an intensity map of the reflected radiation. A processor is in communication with the detector and has software resident thereon for determining from an analysis of the data a pupil size and an intensity level in the intensity map. A controller is in communication with the processor and the beam processor and has means for signaling and causing the beam-altering means to vary the second spot size to optimize an accuracy of the determined pupil size and the intensity level.

The variable spot size that is provided at the retina permits the tracker beam focused on the corneal surface to be projected at the retina with an optimal desired size, so that the pupil glow image formed by the light back-reflected and scattered at the retina forms a pupil glow image at the correct size; that is, with no missing edge rays, and having high signal-to-noise ratio (pupil glow return vs. camera noise), even for subjects with large pupil size and high refractive error.

The embodiments of the system and method of this invention may be used on objects other than corneas, and in surgical procedures other than corneal ablation.

### BRIEF DESCRIPTION OF THE DRAWINGS

FIG. 1 is an exemplary generalized schematic of a system for generating a variable spot size for illuminating a pupil with retinal reflection, and for detecting the reflected radiation in accordance with the teachings of this invention;

FIG. 2 is a specific embodiment of a variable-spot-size generating and detection system of this invention;

FIG. 3 is another embodiment of a variable-spot-size generating and detection system of this invention; and

FIGS. 4A-4I are simulation results of pupil glow images for a model eye with a +5D refractive error and an 8-mm pupil. FIGS. 4A-4D are for a 0° rotation; FIGS. 4E-4I, for a 4° rotation. FIG. 4A, beam collimated at cornea; FIGS. 4B-4D, beam focused at cornea; FIG. 4E, beam collimated at cornea; FIGS. 4F-4I, beam focused at cornea.

### DETAILED DESCRIPTION OF THE PREFERRED EMBODIMENTS

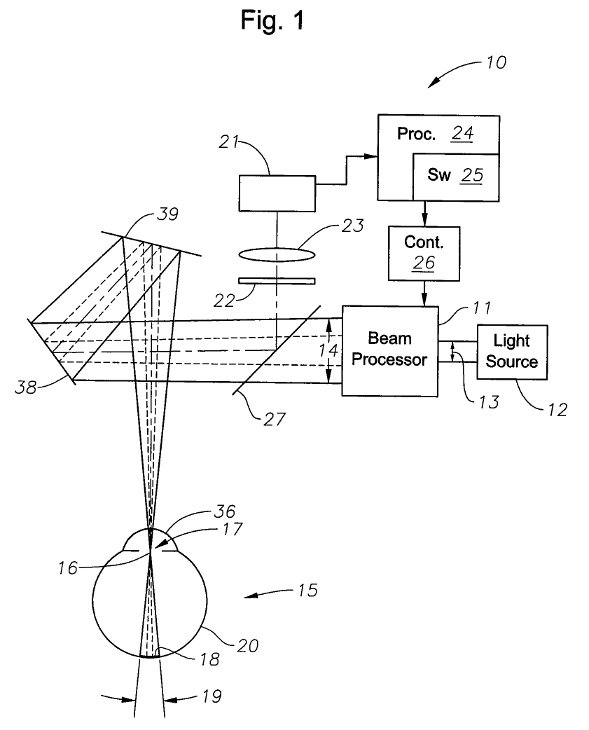

The present invention will now be described with reference to FIGS. 1-41.

A system and method for tracking transverse movement comprise a pupil tracking device that uses “pupil glow” to determine the center of the pupil for the purpose of maintaining an ablating laser beam in a preferred orientation relative to a cornea.

A system 10 for generating a beam for retinal reflection detection comprises, in general (FIG. 1) a beam processor 11 that is positioned to receive an illumination beam from a light source 12. The beam has a first spot size 13. The beam processor 11 comprises means for altering the illumination beam to form a beam having a second spot size 14. The beam processor can comprise, for example, one of a diffractive component, a refractive component, a spatial light modulator, and a micro-electro-mechanical system, as will be understood by one of skill in the art.

Focusing optics are positioned to receive the beam emitted from the beam processor 11 and to focus the emitted beam onto an eye 15, so that a focal point 16 of the emitted beam is adjacent a center of a pupil 17 of the eye 15, and a spot 18 having a spot size 19 is formed on a retina 20 of the eye 15. The emitted beam should be smaller than the pupil size, and should preferably be positioned adjacent the pupil center.

A detector, for example, a CCD or CMOS array 21, is adapted to receive reflected radiation from the retina 20 passed through the pupil 17, and to generate data indicative of a spatial extent of the eye pupil 17 and an intensity map of the reflected radiation. In a particular embodiment, the detector 21 is preceded by a filter 22 and a camera lens 23.

A processor 24 is in communication with the detector 21, and has software 25 resident thereon for determining from an analysis of the data a pupil size and an intensity level in the intensity map. A controller 26 is in communication with the processor 24 and the beam processor 11 that has means for signaling the beam processor 11 to vary the second spot size to optimize an accuracy of the determined pupil size and the intensity level.

The processor 24 (or circuit) may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. The processor 24 may have an associated memory and/or memory element 50, which may be a single memory device, a plurality of memory devices, and/or embedded circuitry of the processor 24. Such a memory device may be a read-only memory, random access memory, volatile memory, non-volatile memory, static memory, dynamic memory, flash memory, cache memory, and/or any device that stores digital information. Note that when the processor 24 implements one or more of its functions via a state machine, analog circuitry, digital circuitry, and/or logic circuitry, the memory and/or memory element 50 storing the corresponding operational instructions (e.g., software 25) may be embedded within, or external to, the circuitry comprising the state machine, analog circuitry, digital circuitry, and/or logic circuitry. Further note that, the memory element 50 stores, and the processor 24 executes, hard coded and/or operational instructions (e.g., software 25) corresponding to at least some of the steps and/or functions disclosed herein and illustrated in FIGS. 1-3.

A beam splitter 27 directs the beam from the beam processor 11 to the eye 15, and also directs the returning beam from the eye 15 to the detector 21.

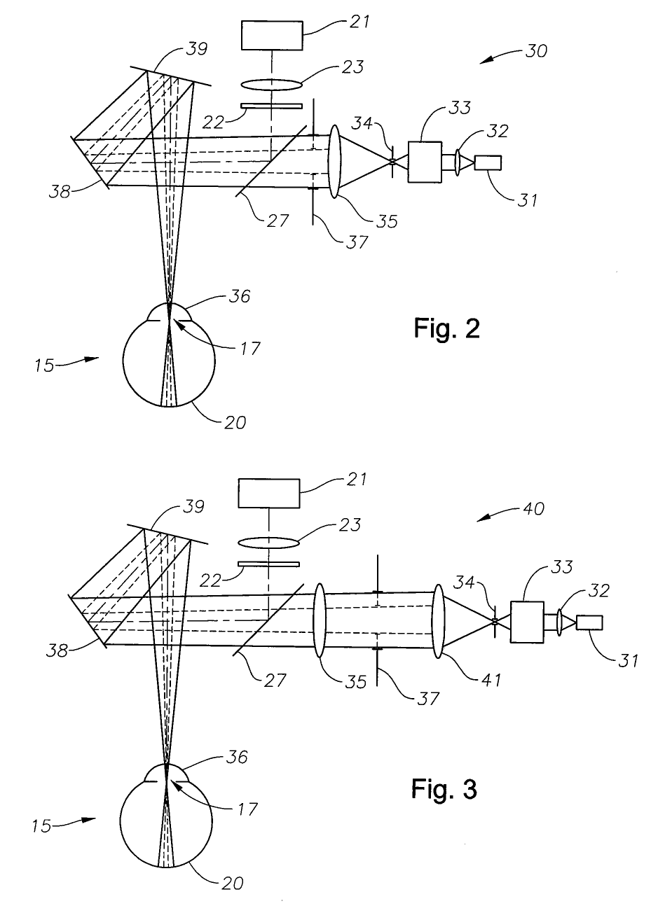

Two specific embodiments 30,40 of the system of the present invention are illustrated in FIGS. 2 and 3, although these are not intended as limitations. In both of these embodiments 30,40 the light source comprises a laser diode 31 emitting in a near-infrared range of 780-1100 nm, and preferably at approximately 905 nm, followed by a collimating lens 32. The beam processor comprises high-numerical-aperture focusing optics 33 for producing a focused beam and a small aperture 34 of diameter 0.1 mm or smaller positioned to receive the focused beam for achieving spatial filtration thereof. The high-numerical-aperture focusing optics 33 can comprise, for example, at least one of a microscope objective, an aspherical lens, a GRIN (gradient index) lens, and a diffractive element.

Scanning optics, such as a pair of scanning mirrors 38,39, are positioned downstream of the beam processor for retaining the emitted beam at a predetermined orientation with respect to the eye 15. The control of the scanning optics is outside the scope of the present invention, but scanning optics control schemes/mechanisms are well known in the art.

In the embodiment 30 of FIG. 2, an imaging lens 35 is positioned downstream of the small aperture 34, for imaging the small aperture 34 on a cornea 36 of the eye 15. The altering means comprises a variable-size aperture 37 downstream of the imaging lens 35, for spatially filtering radiation emerging from the imaging lens 35 to control the spot size at the retina 20.

In the embodiment 40 of FIG. 3, a second collimation lens 41 is positioned downstream of the small aperture 34, and the variable-size aperture 37 is positioned downstream of the collimation lens 41, for spatially filtering radiation emerging from the collimation lens 41 to control the spot size at the retina. An imaging lens 35 is provided downstream of the variable-size aperture 37, for focusing an incoming beam on the cornea 36. Since the distance between the second collimation lens 41 and the imaging lens 35 has essentially no impact on system performance, the system 40 can provide a long optical path if desired, depending upon the design of the entire system.

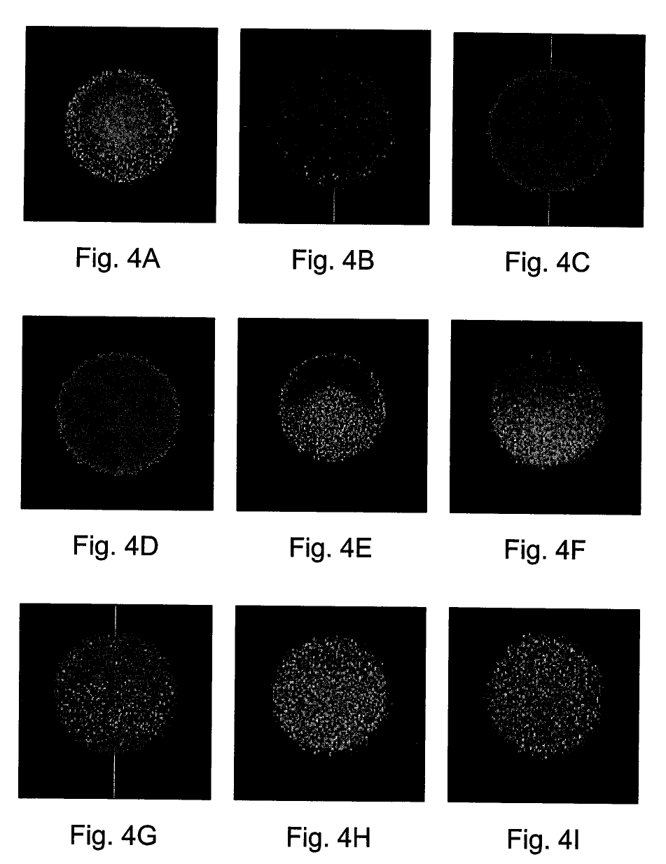

Exemplary simulation results are illustrated in FIGS. 4A-4I, showing the simulated pupil glow images for a model eye with +5D refractive error, 8 mm pupil size, and either 0° or 4° eye rotation. The retina is illuminated by a beam with variable spot size. Table 1 presents the measured pupil size from the pupil glow image for different spot sizes, as well as the normalized intensity relative to the intensity level reached with the focused beam at the retina for an eye coaxial with the optical axis. From these results it can be seen that a smaller spot size at the retina can provide a smaller pupil glow image, but at a relatively higher intensity. When the spot size at the retina is too large, the intensity of the pupil glow image is dramatically decreased. For the simulated case, it can be seen that a 1.3- and 1.5-mm spot size at the retina provides a pupil glow image at the correct size and with an intensity sufficiently high to enable tracking, while a smaller spot size at the retina indicates a significantly smaller pupil size than is correct.

TABLE 1

| Eye rotation | Retinal | | Measured pupil | Normalized relative |
|---|---|---|---|---|
| (deg) | spot size | | size (mm) | intensity level |
| 0 | <50 | μm | 7.64 | 1 |
| 0 | 1 | mm | 7.93 | 0.37 |
| 0 | 1.3 | mm | 8 | 0.23 |
| 0 | 2.0 | mm | 8 | 0.10 |
| 4 | <50 | μm | 7.40 | 0.82 |
| 4 | 1 | mm | 7.76 | 0.25 |
| 4 | 1.3 | mm | 7.85 | 0.21 |
| 4 | 1.5 | mm | 8 | 0.18 |
| 4 | 2.0 | mm | 8 | 0.09 |

Although the invention has been described relative to specific embodiments thereof, there are numerous variations and modifications that will be readily apparent to those skilled in the art in the light of the above teachings. It is therefore to be understood that, within the scope of the appended claims, the invention may be practiced other than as specifically described.

What is claimed is:

1. A system for generating a beam for retinal reflection detection comprising:
a beam processor positioned to receive an illumination beam from a light source, the beam having a first spot size, and the beam processor comprising means for altering the illumination beam to form a beam having a second spot size;
focusing optics positioned to receive the beam emitted from the beam processor and to focus the emitted beam onto an eye, a focal point of the emitted beam adjacent a center of a pupil of the eye, the emitted beam thereby forming a spot on a retina of the eye;
a detector adapted to receive reflected radiation from the retina passed through the pupil, and to generate data indicative of a spatial extent of the eye pupil and an intensity map of the reflected radiation;
a processor in communication with the detector having software resident thereon for determining from an analysis of the data a pupil size and an intensity level in the intensity map; and
a controller in communication with the processor and the beam processor having means for signaling the beam-altering means to vary the second spot size to optimize an accuracy of the determined pupil size and the intensity level.

2. The system recited in claim 1, wherein the beam processor comprises one of a diffractive component, a refractive component, a spatial light modulator, and a micro-electro-mechanical system.

3. The system recited in claim 1, wherein the beam processor comprises:
high-numerical-aperture focusing optics for producing a focused beam;
a small aperture positioned to receive the focused beam for achieving spatial filtration thereof, and
an imaging lens for imaging the small aperture on a cornea of the eye; and
wherein the altering means comprises a variable-size aperture for spatially filtering radiation emerging from the imaging lens to control the spot size at the retina.

4. The system recited in claim 3, wherein the high-numerical-aperture focusing optics comprises at least one of a microscope objective, an aspherical lens, a GRIN lens, and a diffractive element.

5. The system recited in claim 1, wherein the beam processor comprises:
high-numerical-aperture focusing optics for producing a focused beam;
a small aperture positioned to receive the focused beam for achieving a spatial filtration thereof;
a collimation lens positioned downstream of the small aperture; and
an imaging lens for focusing an incoming beam on the cornea;
wherein the altering means comprises a variable-size aperture upstream of the imaging lens and downstream of the collimation lens, for spatially filtering radiation emerging from the collimation lens to control the spot size at the retina.

6. The system recited in claim 1, wherein the detector comprises a charge-coupled-device array.

7. The system recited in claim 1, wherein the beam processor is positioned to receive an illumination beam from a laser diode, and further comprising a collimation lens downstream of the laser diode and upstream of the focusing optics, for delivering a collimated beam to the focusing optics.

8. The system recited in claim 1, further comprising scanning optics for retaining the emitted beam at a predetermined orientation with respect to the eye.

9. A method for generating a beam for retinal reflection detection comprising the steps of:
receiving an illumination beam from a light source, the beam having a first spot size;
altering the illumination beam to form a beam having a second spot size;
focusing the altered beam onto an eye, a focal point of the altered beam adjacent a center of a pupil of an eye, the altered beam thereby forming a spot on a retina of the eye;
detecting reflected radiation from the retina passed through the pupil;
generating data indicative of a spatial extent of the eye pupil and an intensity map of the reflected radiation;
determining from an analysis of the data a pupil size and an intensity level in the intensity map; and
signaling the beam-altering means to vary the second spot size to optimize an accuracy of the determined pupil size and the intensity level.

10. The method recited in claim 9, wherein the altering step is performed using one of a diffractive component, a refractive component, a spatial light modulator, and a micro-electro-mechanical method.

11. The method recited in claim 9, wherein the altering step comprises:
producing a focused beam;
spatially filtering the focused beam using a small aperture;
imaging the small aperture on a cornea of the eye; and
using a variable-size aperture for spatially filtering radiation emerging from the imaging lens to control the spot size at the retina.

12. The method recited in claim 11, wherein the focusing step comprises using at least one of a microscope objective, an aspherical lens, a GRIN lens, and a diffractive element.

13. The method recited in claim 9, wherein the altering step comprises:
producing a focused beam;
spatially filtering the focused beam using a small aperture;
collimating the spatially filtered beam;
spatially filtering the collimated beam to control the spot size at the retina;
focusing the spatially filtered, collimated beam on the cornea.

14. The method recited in claim 9, wherein the detecting step is performed using a charge-coupled-device array.

15. The method recited in claim 9, wherein the light source comprises a laser diode, and further comprising the step of collimating the illumination beam prior to the altering step.

16. The method recited in claim 9, further comprising the step of retaining the emitted beam at a predetermined orientation with respect to the eye.

17. The method recited in claim 16, wherein the retaining step is performed with the use of scanning mirrors between the focusing step and the detecting step.
